(19) United States
(12) Patent Application Publication
(10) Pub. No.: US 2020/0140892 A1
(43) Pub. Date: May 7, 2020

(54) CELL TRANSFER AGENT

(71) Applicants: Toshihiro AKAIKE, Tokyo (JP); Mitsuaki GOTO, Kawasaki-Shi, Kanagawa (JP)

(72) Inventors: Toshihiro AKAIKE, Tokyo (JP); Mitsuaki GOTO, Kawasaki-Shi (JP); Teiko SEKI, Yokohama-shi (JP)

(73) Assignees: Toshihiro AKAIKE, Tokyo (JP); Mitsuaki GOTO, Kawasaki-Shi, Kanagawa (JP)

(21) Appl. No.: 16/617,943

(22) PCT Filed: May 29, 2017
(86) PCT No.: PCT/JP2017/019853

§ 371 Date: Nov. 27, 2019

(51) Int. Cl. C12N 15/87

(52) U.S. Cl. CPC C12N 15/87

(57) ABSTRACT

The present invention is a cell transfer agent comprising a composite particle coated by a sugar chain polymer wherein the composite particle consisting of an apatite comprising phosphate, carbonic acid, and calcium.

### TECHNICAL FIELD

The present invention relates to a cell transfer agent containing composite particles coated with a sugar chain polymer, and the composite particle is composed of apatite containing phosphate, carbonate and calcium.

### BACKGROUND ART

The transformation of DNA into mammalian cells is an extremely effective research technique relating to gene structure, function and control, and is expected in the field of gene therapy, DNA vaccine and the like. As a conventional gene transfer method, a virus method using a recombinant such as a retrovirus and an adenovirus as a vector for gene therapy is common.

However, the virus is pointed out as a problem of its own toxicity, immunogenicity, and the like. Further, problems such as restriction of the size of the applicable gene and a high price are known.

For this reason, development of gene transfer (transfection) technology which does not use a virus instead of a viral vector has been actively carried out for the purpose of basic research and application to gene transfer treatment. As a method for a non-viral gene transfer, there are known various methods such as a calcium phosphate method using a coprecipitate of DNA and calcium, a lipofection method for forming composite particles of a cationic lipid such as liposome and an anionic DNA.

As a cell transfer agent for transferring a target substance such as a polynucleotide into a cell, a cell transfer agent comprising a calcium phosphate-based material is known (patent document 1).

However, these cell transfer agents have disadvantages of lack of biocompatibility.

### PRIOR ART DOCUMENTS

### Patent Documents

Patent Document 1: WO2004/043376

### SUMMARY OF INVENTION

### Technical Problem

The present invention provides a cell transfer agent excellent in biocompatibility.

### Solution to Problem

As a result of intensive studies to solve the above problems, the present inventors have found that a composite particle made of a calcium phosphate-based material can be coated with a sugar chain polymer to impart biocompatibility to the composite particle, thereby completing the present invention.

That is, the present invention is as follows.

[1] A cell transfer agent comprising a composite particle coated with a sugar chain polymer or a phosphorylated sugar chain, wherein the complex particle consists of an apatite containing phosphate, carbonate and calcium.
[2] The cell transfer agent according to [1], wherein a main chain of the sugar chain polymer is polylysine, chitosan, or polyethyleneimine
[3] The cell transfer agent according to [1]or [2], wherein the average particle size of the composite particles is 500 nm or less.
[4] The cell transfer agent according to any one of [1] to [3], wherein a sugar chain terminal introduced into the sugar chain polymer is galactose, glucose, mannose, N-acetylglucosamine, N-acetylgalactosamine, fucose or sialic acid.
[5] The cell transfer agent according to any one of [1] to [4], further comprising boron, fluorine, cesium, or strontium.
[6] The cell transfer agent according to any one of [1] to [5], wherein the pH is 6.0-9.0.
[7] The cell transfer agent according to [1], wherein the phosphorylated sugar chain is one that any one of hydroxyl groups of mannose, glucose, or N-acetylglucosamine is phosphorylated.
[8] The cell transfer agent according to [8], wherein the phosphorylated sugar chain is mannose-6-phosphate, gluccose-6-phosphate, N-acetylglucosamine-6-phosphate.
[9] The cell transfer agent according to [7], wherein the phosphorylated sugar chain is mannose-1-phosphate, glucose-1-phosphate, N-acetylglucosamine-1-phosphate.
[10] A method for producing a cell transfer agent comprising a composite particle coated with a sugar chain polymer or a phosphorylated sugar chain, wherein the composite particle is consisted of an apatite containing phosphate, carbonate and calcium, comprising the step of:
forming the composite particle by preparing a composition comprising at least calcium ion, phosphate ion, and hydrogen carbonate ion in the presence of a sugar chain polymer.
[11] A method for producing a composite particle consisting of an apatite comprising phosphate, carbonate and calcium, wherein the average particle size of the composite particles is 10 nm or less, comprising the step of:
forming the composite particles by preparing a composition comprising a calcium ion, a phosphate ion and a hydrogen carbonate ion, wherein the phosphate ion is PBS of 10 times concentration and
diluting the obtained composite particles to 1/10.
[12] A method for producing composite particles consisting of an apatite comprising phosphate, carbonic acid and calcium, wherein the average particle size of the composite particles is 70-130 nm, comprising the step of:
forming the composite particles by preparing a composition comprising a calcium ion, a phosphate ion, and a hydrogen carbonate ion, wherein the phosphate ion is PBS of 10 times concentration and the step is carried out at from 4° C. to 20 ° C.

### Advantageous Effects of the Invention

A cell transfer agent of the present invention has an effect of excellent biocompatibility.

### BRIEF DESCRIPTION OF THE DRAWINGS

FIG. 1 is a ¹H-NMR spectrum of polylysine-lactobionic acid. 1.3 to 1.8 ppm is a peak derived from polylysine, and 2.7 to 4.2 ppm is a peak derived from sugar chains.

FIG. 2 is a ¹H-NMR spectrum of polylysine-N-acetylglucosamine. 0.3 to 1.8 ppm is a peak derived from polylysine, and 2.7 to 4.2 ppm is a peak derived from a sugar chain.

FIG. 3 is a fluorescent photograph after 24 hours after interacting with carbonate nanoparticles of various sugar chain polymer coatings containing 3T3 cells and a pEGFP-N2 plasmid prepared in the example.

FIG. 4 is a fluorescent photograph after 24 hours after interacting with carbonate nanoparticles of various sugar chain polymer coatings containing Hela cells and a pEGFP-N2 plasmid prepared in the example.

FIG. 5 is a fluorescence photograph after 24 hours after interacting with carbonate nanoparticles of various sugar chain polymer coatings containing HepG2 cells and a pEGFP-N2 plasmid prepared in the example.

FIG. 6 is a fluorescent photograph after 24 hours after interacting with carbonate nanoparticles of various sugar chain polymer coatings containing 3T3 cells and a PT2-RFP plasmid prepared in the example.

The cell transfer agent of the present invention can transfer a target substance into a cell extremely efficiently. The target substance can include, but is not limited to, agents, proteins, and polynucleotides.

The cell transfer agent of the present invention includes composite particles coated with a sugar chain polymer. The composite particles are composed of apatite containing phosphate, carbonic acid, and calcium.

The composite particles of the present invention can be produced by a conventionally known method. For example, the apatite of the present invention can be produced by adding a solution containing a calcium ion to a solution containing phosphate ions and carbonate ions.

In the present invention, the calcium phosphate-based material constituting the composite particle is a material containing ca and po4 as main components. In the invention, the calcium phosphate-based material is preferably apatite. As the apatite, hydroxyapatite, apatite, or the like can be used. In particular, apatite is preferably used.

The hydroxyapatite suitably used in the present invention is represented by Ca₁₀₋ₘXₘ(PO₄)₆(CO₃)₁₋ₙYₙ. Here, X is an element capable of partially substituting ca in the apatite, and for example, Sr, Mn, a rare earth element or the like can be exemplified. M is a positive number of 0 or more and 1 or less, preferably 0 to 0.1, more preferably 0 to 0.01, and particularly preferably 0 to 0.001y is an unit in which CO₃ in apatite can be partially replaced, and OH, F, Cl, and the like can be exemplified. N is a positive number of 0 to 0.1, preferably 0 to 0.01, more preferably 0 to 0.001, and particularly preferably 0 to 0.0001.

The composite particle of the present invention can be coated with a sugar chain polymer by adding a sugar chain polymer to a solution containing the same.

The main chain of the sugar chain polymer of the present invention can be any known polymer. Preferably, the main chain of the sugar chain polymer is polylysine, chitosan, polyglutamic acid or polyethyleneimine.

The average particle size of the composite particles contained in the cell transfer agent of the present invention is preferably 500 nm or less, more preferably 400 nm or less, still more preferably 300 nm or less, and particularly preferably 200 nm or less. The smaller the average particle size of the composite particles, the more the incorporation efficiency of the composite particles into the cells can be improved. The lower limit of the average particle size of the composite particles is not particularly limited, but is usually 1 nm or more.

Any sugar chain known in the art can be used as a sugar chain transferred into the sugar chain polymer of the present invention. The sugar chain is a compound in which various saccharides are linked by a glycoside bond, and the number of bound and the like is two or more. The terminal of the sugar chain transferred into the sugar chain polymer of the present invention is preferably galactose, glucose, mannose, N-acetylglucosamine, n-acetyl galactosamine, fucose or sialic acid.

Specific examples of agents usable in the present invention include anticancer agents and anti-tumor antibiotics. Specific examples of anticancer agents include Methotrexate (anti-folic acid agent), Vinblastine (vinca alkaloid), Anthracycline; Daunomycin, Adriamycin. Anti-tumor antibiotics include Duocarmysin, Enediynes, Necarzinostatin, Calicheamicin, and Macrolides. By forming the composite particles by using such a medicine, the intracellular transfer efficiency of the drug can be improved, so that the composite particle can be suitably used for various kinds of diseases treatment.

As a polynucleotide, any of DNA and RNA can be used, and a hybrid polynucleotide comprising DNA and RNA can also be used. For example, when gene recombination is performed using the cell transfer agent of the present invention, composite particles may be formed by using a vector DNA containing a gene to be expressed. Any DNA such as a cyclic plasmid DNA, a straight-chain plasmid DNA, an artificial chromosome, and a triple-stranded DNA may be used as the DNA. Alternatively, composite particles may be formed by using RNA capable of adjusting the cell function, e.g., antisense RNA, or siRNA causing RNA interference.

The cell transfer agent contains the composite particles. The cell transfer agent of the present invention is not particularly limited in its dosage form as long as it can be transferred into a cell without modifying the target substance, and any type of agent type such as powder, solid matter, solution or the like may be used.

In the present invention, various cells such as bacterial cells, actinomycete cells, yeast cells, fungi cells, plant cells, insect cells, and animal cells can be used as a target for transferring the target substance. Among these, animal cells, especially mammalian cells, can be preferably used. The target cells to be transferred into the target substance are included in in vitro or in vivo. That is, any cell such as cultured cell, cultured tissue, living body or the like may be used

When a cultured cell is used, a medium containing the cell transfer agent of the present invention is prepared, and the culture is carried out using the culture medium under normal culture conditions, whereby the target substance can be transferred into the cell.

When the cell transfer agent of the present invention is used as a pharmaceutical for treating various diseases, for example, a cell transfer agent containing composite particles composed of a substance having a pharmacological activity and a calcium phosphate-based material is prepared, and the cell transfer agent is administered to a subcutaneous or intramuscular, intraperitoneal or blood vessel of a mammal (including a human) to directly transfer a substance having pharmacological activity to the living cell.

When used as a medicine for gene therapy, a cell transfer agent containing a polynucleotide (for example, vector DNA, antisense RNAi, etc.) and a calcium phosphate-based material capable of adjusting a cell function can be prepared, and transferred and expressed into the target cell. Examples of diseases to be subjected to gene therapy include diseases such as cancer or genetic disease.

### EXAMPLES

The present invention is described in more detail below on the basis of examples. The present invention is not limited to the following examples.

A commercially available PBS (Gibco) is prepared from a powder so as to have a concentration of 10 times, and 2.0 g of sodium bicarbonate is dissolved dissolving in 50 ml of the solution to adjust the pH to 7.4. The vector (pT2-GFP) incorporating the expression gene of the GFP is added to make 1 μg/ml solution and incubated for 30 minutes. Then, 5.2 ml of a calcium chloride solution (1M) is added and incubated at 37° C. for 30 minutes. Then, 1 ml of the above solution is added to 9 ml of physiological saline, and the resultant solution is immediately subjected to ultrasonic treatment with a bath type sonicator (US-LOPS, SND) for one minute. Then, the particle size is measured immediately by DLS (Malvern Zetasizer Nano 90 and Otsuka Electronics DLS-1000). When a sugar chain coating is applied, a sugar chain polymer is added in a prescribed amount (5.2 ml) when diluted with physiological saline.

The average value (scattering intensity) of the particle diameter (nm) before dilution of the carbonate nanoparticles prepared from the 10-fold concentration of PBS is 2343.6±4071.0 (peak 1:247.2±154.9) and the peak 2:7775.5±4308.1. On the other hand, after dilution, the average value (number conversion) is 8.5±2.2. As seen above, carbonate nanoparticles having an average particle size of 6 to 11 nm can be easily produced by diluting the carbonate nanoparticles prepared at a high concentration.

A commercially available PBS (Gibco) is prepared from a powder so as to have a concentration of 10 times, and 2.05 g of sodium bicarbonate is dissolved dissolving in 50 ml of the solution to adjust the pH to 7.4. The vector (pT2-GFP) incorporating the expression gene of the GFP is added to make 1 μg/ml solution and incubated for 30 minutes. Then, 5.2 ml of a calcium chloride solution (1M) is added and incubated at 4, 20, and 37° C. for 30 minutes. Then, 1 ml of the above solution is added to 9 ml of physiological saline, and the resultant solution is immediately subjected to ultrasonic treatment with a bath type sonicator (US-10PS, SND) for one minute. Then, the particle size is measured immediately by DLS (Malvern Zetasizer Nano 90 and Otsuka Electronics DLS-1000). When a sugar chain coating is applied, a sugar chain polymer is added in a prescribed amount (5.2 ml) when diluted with physiological saline.

Whereas the average value (scattering intensity) of the particle diameter (nm) before dilution of the carbonate nanoparticles prepared from the 10-fold concentration of PBS is 1170 to 1390 nm as number conversion produced at 37° C., the average value (scattering intensity) is 72.2 to 106 nm at 20° C., and it is 101 to 127 nm at 4° C. From this, it is possible to prepare carbonate nanoparticles having an average particle size of 70 to 130 nm by being prepared at a low temperature.

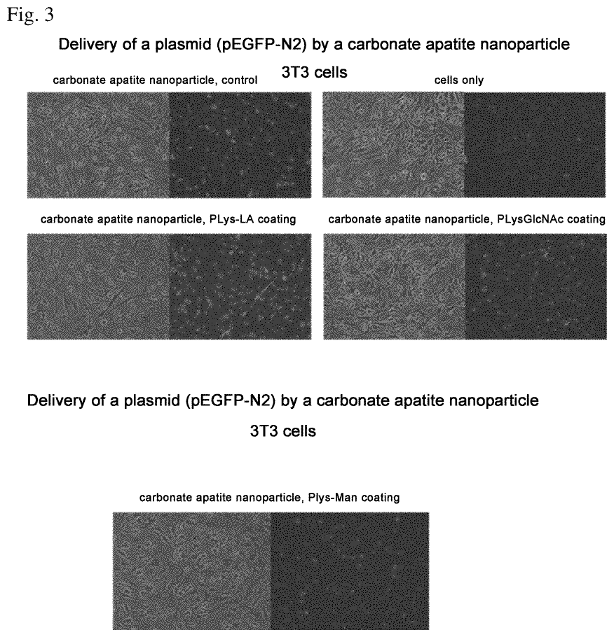

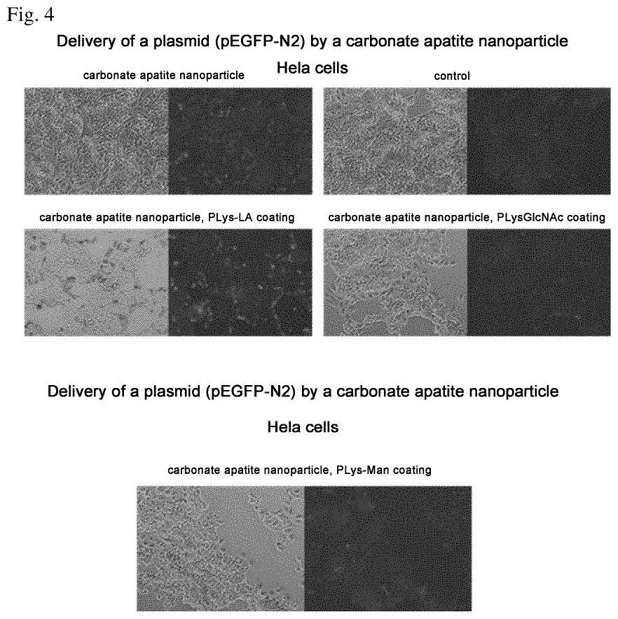

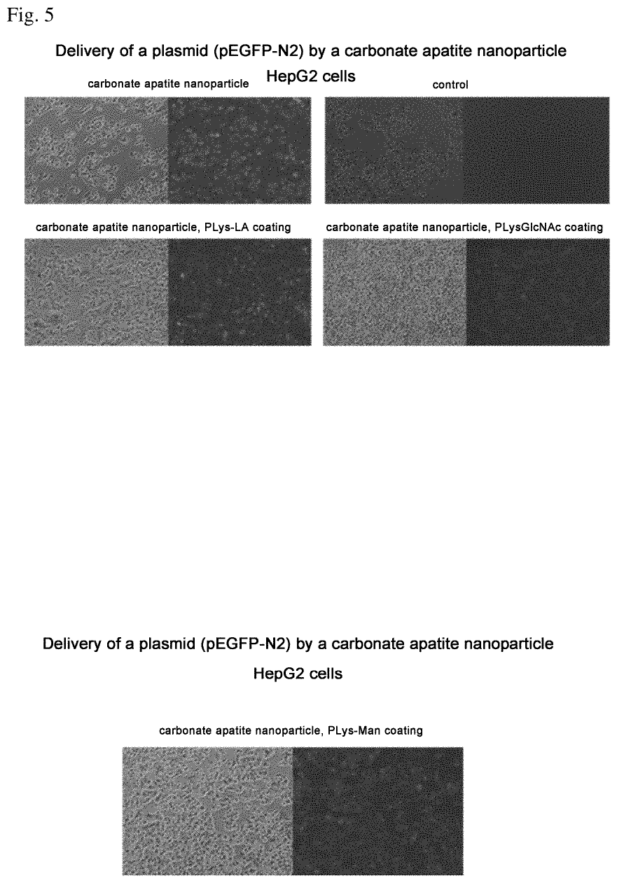

185 mg (2.2 mmol) of sodium bicarbonate is added to 50 ml of commercial DMEM, and pH is adjusted to 7.4. A vector incorporating the expression gene of the GFP (pT2-GFP, or pT2-RFP or pEGFP-N2) is added to make the concentration of 1 μg/ml and incubated for 30 minutes. Then, 5 μl of a calcium chloride solution is added to 1 ml of the solution, and the solution is incubated at 37° C. for 30 minutes. 100 μl of the solution is added to a cell culture dish (6 well dish, cell number 1×10⁵/ml) and the expression amount of GFP is quantified after overnight culture. When a sugar chain coating is applied, a prescribed amount (5 μl) of a sugar chain polymer is added before the addition of calcium chloride. The results are shown in FIGS. 3-5.

In any of 3T3 cells, Hela cells, and HepG2 cells from this data, there is a difference in the transfer of the plasmid into the cells depending on the sugar chains, and the emission of GFP in the cells is different. In particular, it is found that the increase in lactose and N-acetylglucosamine, but it is decreased by mannose. It is understood that it is different between sugar chains. Therefore, it has become apparent that sugar chain recognition in cells and uptake of subsequent carbonate nanoparticles are changed.

A commercially available PBS powder (Gibco) is prepared so as to have a concentration of 10 times, and the pH is adjusted to 7.4 by dissolving 2.0 g of sodium bicarbonate in 50 ml of the solution. The vector (pT2-GFP) incorporating the expression gene of the GFP is 1 μg/ml and incubated for 30 minutes. Then, 5.2 ml of a calcium chloride solution (1M) is added and incubated at 37° C. for 30 minutes. Then, 1 ml of the above solution is added to 9 ml of pure water, and the resultant solution is immediately subjected to ultrasonic treatment for one minute by a bath type sonicator (US-LOPS, SND). Then, the particle size is measured immediately by DLS (Malvern Zetasizer Nano 90 and Otsuka Electronics DLS-1000). When a sugar chain coating is applied, a sugar chain polymer is added in a prescribed amount (5.2 ml) when diluted with pure water.

A commercially available PBS powder (Gibco) is prepared so as to have a concentration of 10 times, and the pH is adjusted to 7.4 by dissolving 2.0 g of sodium bicarbonate in 50 ml of the powder. The vector (pT2-GFP) incorporating the expression gene of the GFP is 1 μg/ml and incubated for 30 minutes. Then, 5.2 ml of a calcium chloride solution (20 mM) is added and incubated at 37° C. for 30 minutes. Then, 1 ml of the above solution is added to 9 ml of pure water, and the resultant solution is immediately subjected to ultrasonic treatment for 10 minutes by a bath type sonicator (USB -10 PS, SMT). Then, 5 ml of the prepared solution and 5 ml of PLys-LA (0.0001, 0.001, 0.005, and 0.01%) are mixed, and the particle size of the mixture of time-dependent change (after 0, 5, 10, and 15 minutes) is measured by DLS (Malvern Zetasizer Nano 90 and Otsuka Electronics DLS-1000). As a result, when the polymer concentration is 0.005% or more, carbonate nanoparticles having an average particle diameter of 20 nm or less can be produced (Table 1). Regarding the particle size, the same results are obtained by using other polymers.

TABLE 1

| Polymer concentration | Time (min.) | Particle diameter (nm) |
|---|---|---|
| 0.01% | 0 | 11.2 |
| | 5 | 13.9 |
| | 10 | 12.4 |
| | 15 | 12.1 |
| 0.005% | 0 | 18.2 |
| | 5 | 11.3 |
| | 10 | 11.4 |
| | 15 | 13.7 |
| 0.001% | 0 | 346 |
| | 5 | 45.3 |
| | 10 | 405 |
| | 15 | 250 |
| 0.0001% | 0 | 368 |
| | 5 | 351 |
| | 10 | 638 |
| | 15 | 79 |

A commercially available PBS powder (Gibco) is prepared so as to have a concentration of 10 times, and the pH is adjusted to 7.4 by dissolving 2.0 g of sodium bicarbonate in 50 ml of the solution. The vector (pT2-GFP) incorporating the expression gene of the GFP is added so as to have a concentration of 1 μg/ml and incubated for 30 minutes. Then, 5.2 ml of a calcium chloride solution (20 mM) is added and incubated at 37° C. for 30 minutes. Then, 5 ml of the prepared solution and 5 ml of PLys-LA (0.0001, 0.001, 0.005, and 0.01%) are mixed, and the particle size of the mixture of time-dependent change (after 0, 5, 10, 30, and 35 minutes) is measured by DLS (Malvern Zetasizer Nano 90 and Otsuka Electronics DLS-1000). In the case of applying a sugar chain coating, 5 ml of the prepared solution and 5 ml of PLys-LA (0.005%) are mixed, and the particle size of the mixture of time-dependent change (after 15, 20, 25, 45, and 50 minutes) is measured by DLS (Malvern Zetasizer Nano 90 and Otsuka Electronics DLS-1000). As a result,

TABLE 2

| Presence of Polymer | Time (min.) | Particle diameter (nm) |
|---|---|---|
| with polymer | 0 | 154 |
| | 5 | 285 |
| | 10 | 155 |
| | 30 | 305 |
| | 35 | 257 |
| without polymer | 15 | 10.7 |
| | 20 | 3.07 |
| | 25 | 8.58 |
| | 45 | 30 |
| | 50 | 37.5 |

185 mg (2 2 mmol) of sodium bicarbonate is added to 50 ml of commercial DMEM, and pH is adjusted to 7.4. The vector (pT2-RFP) incorporating the expression gene of the GFP is 1 μg/ml and incubated for 30 minutes. Then, 5 μl of a calcium chloride solution is added to 1 ml of the solution, and the solution is incubated at 37° C. for 30 minutes. 100 μl of this solution is added to a cell culture dish (6-well dish, cell number 1×10⁵/ml) cultured at a prescribed number of cells, and then cultured for overnight, and the expression amount of the RFP is quantified. When a sugar chain coating is applied, a prescribed amount (5 μl) of a sugar chain polymer is added before the addition of calcium chloride or after the addition of calcium chloride. Similarly, phosphorylated saccharides such as mannose-6-phosphate are coated at a concentration of 2.2 mM.

When a Plys-sugar coat is added after 20 mM CA is added, apatite of 300 to 600 nm is formed. When PLys-sugar coating is carried out before 20 mM CA is added, an apatite of 30 to 50 nm is formed.

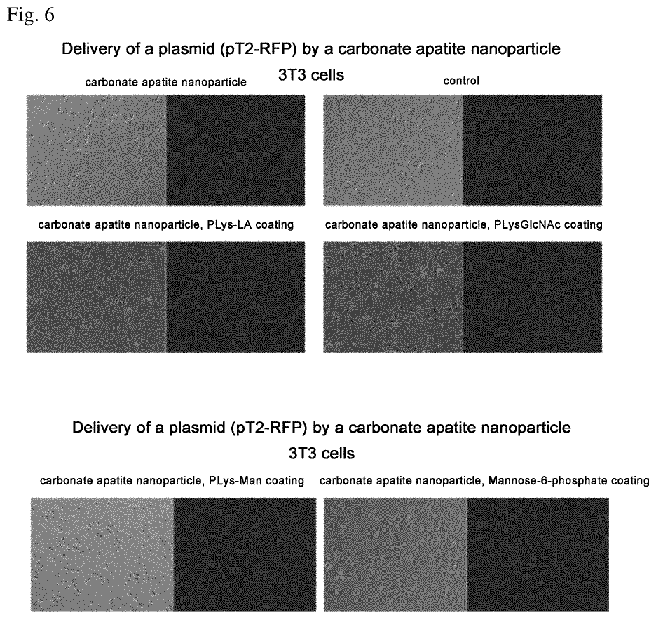

It can be seen from FIG. 6 that incorporation into 3T3 cells is changed in response to sugar chain recognition, and a sugar chain is coated and incorporated into the apatite nanoparticles. Similarly, mannose-6-phosphate-coated carbonate nanoparticles in which the sugar chain is recognized are also incorporated into cells.

1 g of poly-1-lysine having various molecular weights (Sigma-Aldrich) is dissolved in 10 ml of a TEMED buffer (10 mM, pH 4.0) to prepare an aqueous solution. 500 mg of lactobionic acid is added thereto, followed by stirring for 30 minutes, and 500 mg (Tokyo Kasei) of EDC is added thereto. Then, the mixture is stirred and reacted for 3 days. The obtained sugar chain polymer is dialyzed against pure water (60 L), and then freeze-dried to obtain an objective substance.

The synthesis is carried out according to the method of JP-H07-90080.

The sugar chain is synthesized in the same manner as described above using a dimer or derivative of galactose, glucose, mannose, N-acetylglucosamine, N-acetylgalactosamine to obtain an objective substance.

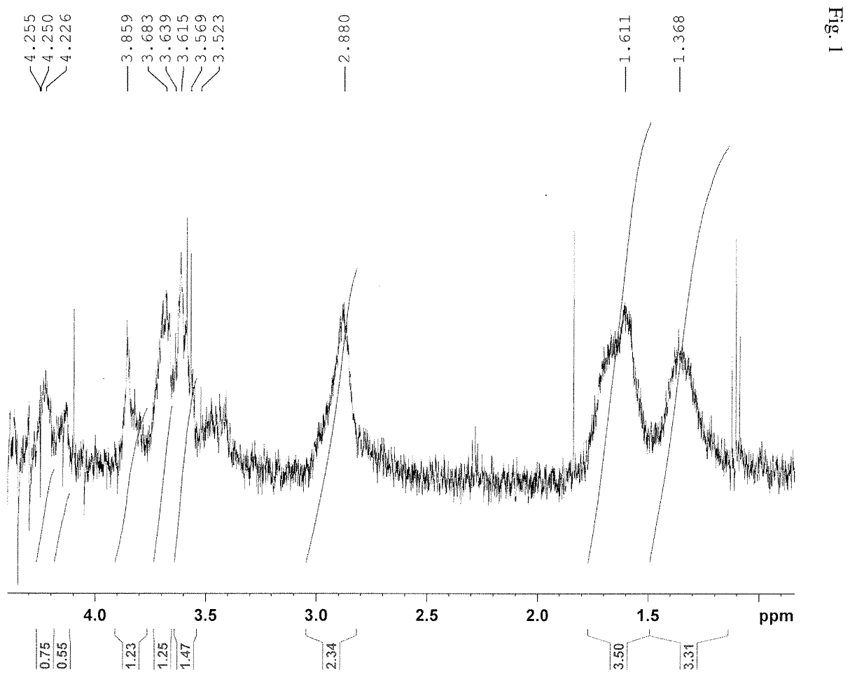

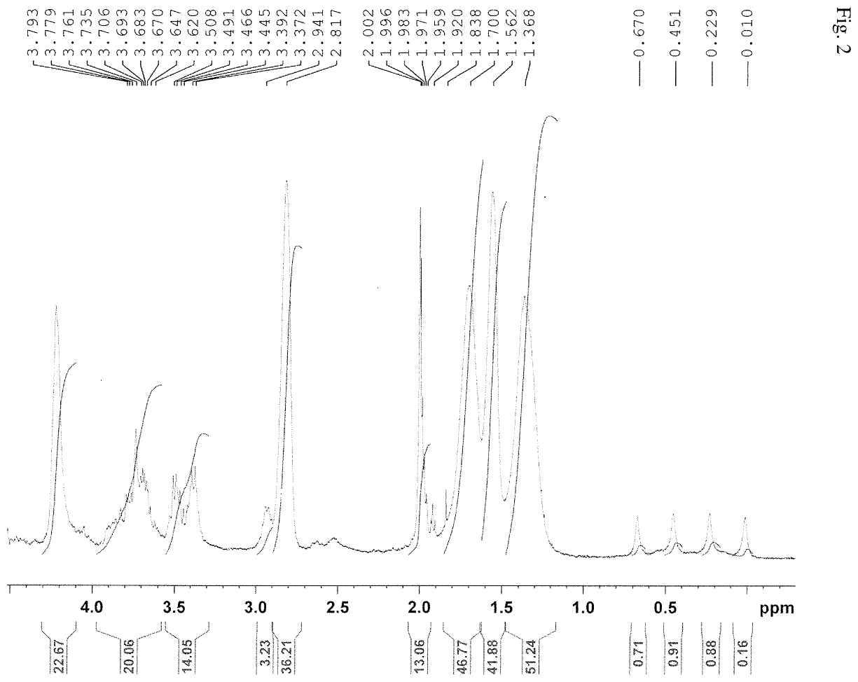

1 g of poly-1-lysine having various molecular weights (Sigma-Aldrich) is dissolved in 10 ml of boric acid buffer (100 mM, pH 8.0) to prepare an aqueous solution. 200 mg of lactobionic acid is added thereto, followed by stirring for two days, and 200 mg of sodium cyanoboric sodium (Wako) is added. Then, the mixture is stirred and reacted for 3 days. The obtained sugar chain polymer was dialyzed against 60 L of pure water, and then freeze-dried to obtain a target (FIGS. 1 and 2).

The sugar chain is synthesized in the same manner as described above using a dimer or derivative of galactose, glucose, mannose, N-acetylglucosamine, N-acetylgalactosamine to obtain an objective substance.

1 g of polyethylene imine having various molecular weights (Sigma-Aldrich) is dissolved in 10 ml of boric acid buffer (100 mM, pH 8.0) to prepare an aqueous solution. 200 mg of lactobionic acid is added thereto, followed by stirring for two days, and 200 mg of sodium cyanoboric sodium (Wako) is added. Then, the mixture is stirred and reacted for 3 days. The obtained sugar chain polymer is dialyzed against pure water (60 L), and then freeze-dried to obtain an objective substance.

The sugar chain is synthesized in the same manner as described above using a dimer or derivative of galactose, glucose, mannose, N-acetylglucosamine, N-acetylgalactosamine to obtain an objective substance.

1 g of chitosan (Sigma-Aldrich) having various molecular weights is dissolved in 10 ml of a TEMED buffer (10 mM, pH 4.0) to prepare an aqueous solution. 500 Mg of lactobionic acid is added thereto, followed by stirring for 30 minutes, and 500 mg (Tokyo Kasei) of EDC is added thereto. Then, the mixture is stirred and reacted for 3 days. The obtained sugar chain polymer is dialyzed against pure water (60 L), and then freeze-dried to obtain an objective substance.

The sugar chain is synthesized in the same manner as described above using a dimer or derivative of galactose, glucose, mannose, N-acetylglucosamine, N-acetylgalactosamine to obtain an objective substance.

Sodium bicarbonate (0.185 g) is added to the PBS (50 mL) to adjust the pH to pH 7.4. Polylysine-LA (lactose-binding polylysine) is added thereto so as to have a final concentration of 0.01, 0.001, and 0.0001 w/v % and a prescribed amount of a calcium chloride solution is added thereto, the resultant mixture is immediately subjected to ultrasonic treatment for one minute by a bath type sonicator (US-LOPS, SND). Then, the particle size is measured immediately by DLS (Malvern Zetasizer Nano 90 and Otsuka Electronics DLS-1000).

### INDUSTRIAL APPLICABILITY

The cell transfer agent of the present invention is useful for transferring a target substance into a cell.

1. A cell transfer agent comprising a composite particle coated with a sugar chain polymer or a phosphorylated sugar chain, wherein the complex particle consists of an apatite containing phosphate, carbonate and calcium.

2. The cell transfer agent according to claim 1, wherein a main chain of the sugar chain polymer is polylysine, chitosan, or polyethyleneimine.

3. The cell transfer agent according to claim 1, wherein the average particle size of the composite particles is 500 nm or less.

4. The cell transfer agent according to claim 1, wherein a sugar chain terminal introduced into the sugar chain polymer is galactose, glucose, mannose, N-acetylglucosamine, N-acetylgalactosamine, fucose or sialic acid.

5. The cell transfer agent according to claim 1, further comprising boron, fluorine, cesium, or strontium.

6. The cell transfer agent according to claim 1, wherein the pH is 6.0-9.0.

7. The cell transfer agent according to claim 1, wherein the phosphorylated sugar chain is one that any one of hydroxyl groups of mannose, glucose, or N-acetylglucosamine is phosphorylated.

8. The cell transfer agent according to claim 7, wherein the phosphorylated sugar chain is mannose-6-phosphate, gluccose-6-phosphate, N-acetylglucosamine-6-phosphate.

9. The cell transfer agent according to claim 7, wherein the phosphorylated sugar chain is mannose-1-phosphate, glucose-1-phosphate, N-acetylglucosamine-1-phosphate.

10. A method for producing a cell transfer agent comprising a composite particle coated with a sugar chain polymer or a phosphorylated sugar chain, wherein the composite particle is consisted of an apatite containing phosphate, carbonate and calcium, comprising the step of:
forming the composite particle by preparing a composition comprising at least calcium ion, phosphate ion, and hydrogen carbonate ion in the presence of a sugar chain polymer.

11. A method for producing a composite particle consisting of an apatite comprising phosphate, carbonate and calcium, wherein the average particle size of the composite particles is 10 nm or less, comprising the step of:
forming the composite particles by preparing a composition comprising a calcium ion, a phosphate ion and a hydrogen carbonate ion, wherein the phosphate ion is PBS of 10 times concentration and
diluting the obtained composite particles to 1/10.

12. A method for producing composite particles consisting of an apatite comprising phosphate, carbonic acid and calcium, wherein the average particle size of the composite particles is 70-130 nm, comprising the step of:
forming the composite particles by preparing a composition comprising a calcium ion, a phosphate ion, and a hydrogen carbonate ion, wherein the phosphate ion is PBS of 10 times concentration and the step is carried out at from 4° C. to 20° C.
